(19) United States
(12) Patent Application Publication
(10) Pub. No.: US 2009/0005616 A1
(43) Pub. Date: Jan. 1, 2009

(54) Purification of 1,2,3,3,3-Pentafluoropropene by Extractive Distillation

(76) Inventors: Ralph Newton Miller, Newark, DE (US); Shoibal Banerjee, Chadds Ford, PA (US); Velliyur Nott Mallikarjuna Rao, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/160,853

(22) PCT Filed: Mar. 29, 2007
(86) PCT No.: PCT/US07/08201

§ 371 Date: Jul. 14, 2008

Related U.S. Application Data
(60) Provisional application No. 60/787,842, filed on Mar. 31, 2006.

(51) Int. Cl. C07C 19/08; C07C 17/383

(52) U.S. Cl. 570/134; 570/178

(57) ABSTRACT

A process for separating 1,2,3,3,3-pentafluoropropene from a first mixture comprising 1,2,3,3,3-pentafluoropropene and 1,1,3,3,3-pentafluoropropene is disclosed. The process involves (a) contacting the first mixture with at least one extractive agent to form a second mixture; (b) distilling the second mixture; and (c) recovering 1,2,3,3,3-pentafluoropropene substantially free of 1,1,3,3,3-pentafluoropropene. The extractive agent used with the present invention increases or decreases the volatility of 1,2,3,3,3-pentafluoropropene or 1,1,3,3,3-pentafluoropropene relative to each other. Also disclosed is a substantially pure 1,2,3,3,3-pentafluoropropene composition.

### BACKGROUND OF THE INVENTION

1. Field of the Invention

The present invention relates to an extractive distillation process for purifying 1,2,3,3,3-pentafluoropropene. The present invention also relates to a substantially pure 1,2,3,3,3-pentafluoropropene composition.

2. Description of Related Art

Halogenated compounds have been widely used in the industry as refrigerants, solvents, cleaning agents, foam blowing agents, aerosol propellants, heat transfer media, dielectrics, fire extinguishing agents, sterilants and power cycle working fluids, et al. However, a number of bromine-containing or chlorine-containing halocarbons are considered to be detrimental toward the Earth's ozone layer. There is a worldwide effort to develop materials having lower ozone depletion and global warming potential that can serve as effective replacements. For example, the hydrofluorocarbon, 1,1,1,2-tetrafluoroethane (HFC-134a) is being used as a replacement for dichlorodifluoromethane (CFC-12) in refrigeration systems. However, HFC-134a has a high global warming potential.

There is a need for manufacturing processes that provide halogenated hydrocarbons that have lower ozone depletion and global warming potentials. The production of hydrofluoroolefins (i.e., unsaturated compounds containing only carbon, hydrogen and fluorine), has been the subject of recent interest to provide environmentally desirable products for use as effective replacements for the existing halogenated compounds. For example, 1,2,3,3,3-pentafluoropropene (HFC-1225ye), having zero ozone depletion and low global warming potentials, has been identified as a potential replacement for existing halogenated compounds.

Purification is an important step in manufacturing these compounds. Conventional distillation is typically used to separate desired products from impurities; however, conventional distillation becomes ineffective when the desired compound has a boiling point close to that of one or more of the impurities. For example, manufacturing of HFC-1225ye by the dehydrofluorination of CF₃CHFCHF₂ (HFC-236ea) can result in the formation of an isomeric contaminant, 1,1,3,3,3-pentafluoropropene (HFC-1225zc). The boiling point of HFC-1225ye is −19.4° C., and the boiling point of HFC-1225zc is −21.8° C. Since the boiling points of HFC-1225ye and HFC-1225zc are very close, their separation by conventional distillation is difficult.

There is a need to develop other purification processes for the production of hydrofluoroolefins.

### BRIEF SUMMARY OF THE INVENTION

The present inventors have found that HFC-1225ye and HFC-1225zc can be separated from each other in the presence of an extractive agent that increases or decreases the volatility of HFC-1225ye or HFC-1225zc relative to each other. That is to say, the extractive agent increases the volatility of 1225ye with respect to 1225zc, or to put it another way, the extractive agent decreases the volatility of 1225zc with respect to 1225ye. Alternatively, depending on the extractive agent, the extractive agent can decrease the volatility of 1225ye with respect to 1225zc, or to put it another way, the extractive agent increases

Extractive distillation processes of a mixture containing HFC-1225zc and HFC-1225ye by using such extractive agents can afford HFC-1225ye products that are substantially free of HFC-1225zc. The problems encountered upon conventional distillation of HFC-1225ye/HFC-1225zc, such as the need for taller and larger diameter columns, higher energy input, and lower resultant HFC-1225ye recovery, can be solved by practicing the present inventive extractive distillation processes.

This invention provides a process for separating HFC-1225ye from a first mixture comprising HFC-1225ye and HFC-1225zc by using at least one extractive agent. The process comprises the steps of (a) contacting said mixture with at least one extractive agent to form a second mixture; (b) distilling said second mixture; and (c) recovering HFC-1225ye substantially free of HFC-1225zc. The extractive agents in this invention may be compounds having a normal boiling point between −10° C. and 120° C. The extractive agents may be selected from a group consisting of cyclic hydrocarbon ethers, non-cyclic hydrocarbon ethers, alcohols, toluene, fluorobenzene, and ketones.

### BRIEF DESCRIPTION OF THE DRAWING(S)

FIG. 1 is a schematic diagram of an extractive distillation system that can be used for practicing an aspect of the present process.

### DETAILED DESCRIPTION OF THE INVENTION

According to the present invention, there is provided a process for separating HFC-1225ye from a first mixture comprising HFC-1225ye and HFC-1225zc by using extractive agents. The process comprises the steps of (a) contacting the first mixture with at least one extractive agent to form a second mixture; (b) distilling the second mixture; and (c) recovering HFC-1225ye substantially free of HFC-1225zc.

By substantially free or substantially pure, it is meant that the HFC-1225ye product contains less than about 100 parts per million by weight (ppm) of HFC-1225zc, and preferably less than about 10 ppm of HFC-1225zc, and more preferably less than about 1 ppm of HFC-1225zc. By impurity is meant any fluorinated compounds other than the HFC-1225ye that may be present in the HFC-1225ye product.

HFC-1225ye as used herein refers to the isomers, E-HFC-1225ye (CAS Reg No. [5595-10-8]) or Z-HFC-1225ye (CAS Reg. No. [5528-43-8]), as well as any combinations or mixtures of such isomers.

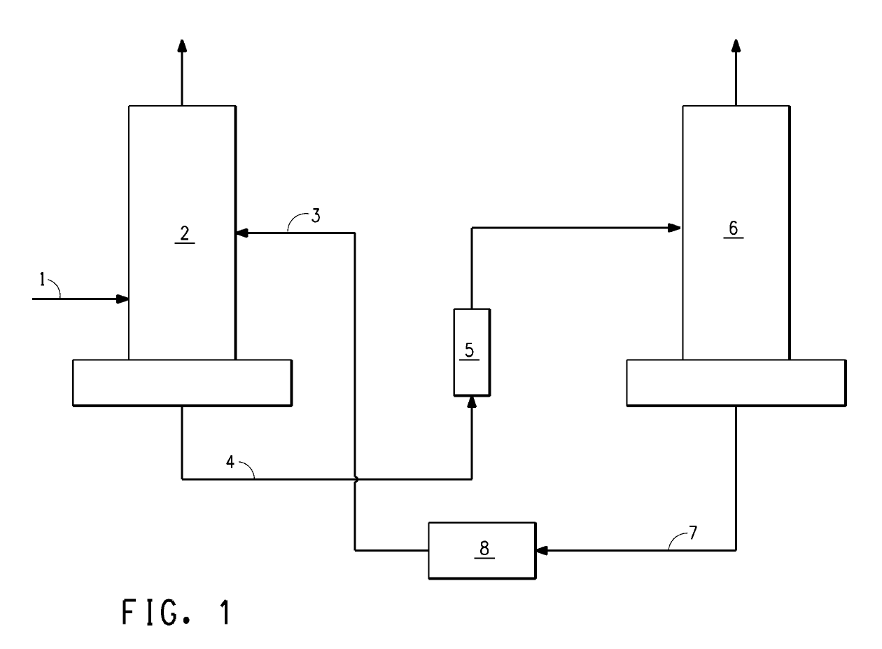

The present inventive process can be better understood by reference to FIG. 1. FIG. 1 schematically illustrates a system which can be used for performing the embodiments of the present extractive distillation process wherein HFC-1225ye is separated from a first mixture comprising HFC-1225ye and HFC-1225zc using at least one extractive agent.

A first mixture comprising HFC-1225ye and HFC-1225zc is supplied via conduit 1 to extraction column 2. At least one extractive agent is supplied via conduit 3 to the extraction column 2 at a feed point higher in the column than the feed point of the first mixture. A stream comprising the extractive agent and HFC-1225ye substantially free of HFC-1225zc is removed from the bottom of column 2 via conduit 4 and transported to optional cooler 5 and from there fed to stripping column 6. The overhead distillate from column 2 contains concentrated HFC-1225zc impurity.

Stripping column 6 separates the extractive agent from HFC-1225ye. Extractive agent is removed from the bottom of column 6 via conduit 7 and transported to optional cooler 8 and from there returned to extraction column 2 as extractant feed. The overhead distillate from column 6 contains HFC-1225ye substantially free of HFC-1225zc and the extractive agent.

In one embodiment, the extractive agents are selected from the group consisting of cyclic hydrocarbon ethers, non-cyclic hydrocarbon ethers, alcohols, toluene, fluorobenzene and ketones.

Cyclic hydrocarbon ethers used as extractive agents with the present invention have from 2 to 6 carbon atoms. Cyclic hydrocarbon ethers in this invention denotes cyclic ethers consisting of C, H and O, wherein the number of carbon atoms is from 2 to 6. Examples of these compounds include tetrahydrofuran (THF), ethylene oxide, propylene oxide (1,2-epoxypropane), oxetane (trimethylene oxide) and tetrahydropyran.

Non-cyclic hydrocarbon ethers used as extractive agents with the present invention have the formula CₓH₂ₓ₊₁OC_{y}H_{2y+1} wherein x and y are 1 or greater and x+y is from 3 to 6. Examples of these compounds include diethyl ether (DEE), dipropyl ether and butyl methyl ether.

Alcohols used as extractive agents with the present invention have the formula C_{z}H_{2z+1}OH wherein z is from 1 to 4. Examples of these compounds include methanol, ethanol, n-propanol, and iso-propanol.

Ketones used as extractive agents with the present invention have the formula CₘH₂ₘ₊₁C(O)CₙH₂ₙ₊₁ wherein m and n are 1 or greater and m+n is at most 5. Examples of these compounds include acetone and 2-butanone (MEK).

In one embodiment of the invention, the extractive agent is selected from the group consisting of tetrahydrofuran, ethylene oxide, propylene oxide, oxetane, tetrahydropyran, diethyl ether, dipropyl ether, butyl methyl ether, methanol, ethanol, n-propanol, iso-propanol, toluene, fluorobenzene, acetone and 2-butanone. In another embodiment of the invention, the extractive agent is selected from the group consisting of methanol, ethanol, n-propanol, iso-propanol. In one preferred embodiment of the invention, the extractive agent is methanol. In another preferred embodiment of the invention, the extractive agent is selected from the group consisting of tetrahydrofuran and propylene oxide. All of the extractive agents and combinations of extractive agents listed hereinabove increase the volatility of HFC-1225zc relative to HFC-1225ye.

In one embodiment of the invention, extractive agents are compounds having a normal boiling point between −10° C. and 120° C. Normal boiling point is defined as the boiling temperature of a liquid at which vapor pressure is equal to one atmosphere. In another embodiment of the invention, extractive agents are compounds having a normal boiling point between 10° C. and 100° C. In yet another embodiment of the invention, extractive agents are compounds having a normal boiling point between 30° C. and 70° C.

The extractive agents according to the present invention may be used alone or in combination with each other as the extractants for the separation. In either case, the extractive agent increases or decreases the volatility of HFC-1225ye or HFC-1225zc relative to each other.

In conventional distillation, only the relative volatilities of the components of the mixture to be separated are used to separate the components. In contrast, the present invention uses extractive distillation. By extractive distillation is meant a process in which an extractive agent is introduced at an upper feed point of a distillation column, whereas the mixture requiring separation is introduced at the same point or preferably, at a relatively lower feed point of the column. The substantially liquid extractive agent passes downwardly through trays or packing in the column and exits the column bottoms with one or more components of the mixture to be separated. While in the presence of the extractive agent, at least one of the components of an initial mixture to be separated becomes relatively more volatile compared to the other components of the mixture, with that more volatile component of the initial mixture exiting the column overheads. Extractive distillation may be employed when the components of a mixture have close relative volatilities that do not afford effective separation of the components by conventional distillation. In extractive distillation, at least one extractive agent is used which causes the relative volatilities of the components in a mixture to be altered such that the resultant relative volatilities, i.e., that of components of the mixture in the presence of the extractive agent, become sufficiently different to permit separation of the components by distillation techniques.

Relative volatility of a chemical compound in a mixture with other compounds is the vapor mole fraction of that compound divided by the liquid mole fraction of that compound. In one embodiment of the invention, 1,2,3,3,3-pentafluoropropene is Z-1,2,3,3,3-pentafluoropropene. In this embodiment, the relative volatility of Z-HFC-1225ye in a mixture with THF is the vapor mole fraction of Z-HFC-1225ye divided by liquid mole fraction of Z-HFC-1225ye.

To determine the relative volatility of a given compound in a mixture with the other compound, a method known as the PTx Method may be used. In this procedure, the total absolute pressure in a cell of known volume is measured at a constant temperature for various compositions of the two compounds. Use of the PTx Method is described in greater detail in “Phase Equilibrium in Process Design”, Wiley-Interscience Publisher, 1970, written by Harold R. Null, on pages 124 to 126; hereby incorporated by reference.

These measurements can be converted into equilibrium vapor and liquid compositions in the PTx cell by using an activity coefficient equation model, such as the Non-Random, Two-Liquid (NRTL) equation, to represent liquid phase nonidealities. Use of an activity coefficient equation, such as the NRTL equation is described in greater detail in “The Properties of Gases and Liquids,” 4^{th} edition, published McGraw Hill, written by Reid, Prausnitz and Poling, on pages 241 to 387, and in “Phase Equilibria in Chemical Engineering,” published by Butterworth Publishers, 1985, written by Stanley M. Walas, pages 165 to 244.

Without wishing to be bound by any theory or explanation, it is believed that the NRTL equation, together with the PTx cell data, can sufficiently predict the ratio of relative volatility of Z-HFC-1225ye and HFC-1225zc, and can therefore predict the behaviour of Z-HFC-1225ye and HFC-1225zc in multi-stage separation equipment such as distillation columns.

The results of PTx measurements and the above calculations indicate that the ratio of relative volatility of Z-HFC-1225ye and HFC-1225zc is close to 1. Thus it is difficult, if not impossible, to separate Z-HFC-1225ye from HFC-1225zc by conventional distillation.

The relative volatilities and their ratios resulting from PTx measurements and the aforementioned calculations for Z-HFC-1225ye and HFC-1225zc in the presence of various extractive agents are summarized in Table 1. Shown are relative volatilities and their ratios at 0° C. for Z-HFC-1225ye/HFC-1225zc at infinite dilution in the listed extraction agent. Also shown are relative volatilities and their ratio at 0° C. for Z-HFC-1225ye/HFC-1225zc without extraction agents.

TABLE 1
Extractive Agents for Z-HFC-1225ye/HFC-1225zc

| | | | Relative Volatility in | | |
|---|---|---|---|---|---|
| | | | Extractive Solvent at 0° C. | | |
| Extractive | | NBP* | | Z-HFC- | |
| Agent | Formula | (° C.) | HFC-1225zc | 1225ye | Ratio |
| THF | (CH₂)₄═O | 66.0 | 64.78 | 39.49 | 1.64 |
| Methanol | CH₃OH | 64.6 | 451.39 | 295.50 | 1.53 |
| None | | | 35.14 | 32.05 | 1.10 |

*NBP = Normal Boiling Point (temperature at which vapor pressure is equal to 1 atmosphere)

As shown above in Table 1, the present inventors have found that the ratio of the relative volatilities of HFC-1225zc to Z-HFC-1225ye can be increased in the presence of the extractive agents. For example, for THF specifically, the volatility of HFC1225zc is increased with respect to the volatility of 1225ye. This discovery allows for separation of HFC-1225ye from a first mixture comprising HFC-1225ye and HFC-1225zc by extractive distillation in the presence of an appropriate extractive agent. The appropriate extractive agent for a first mixture comprising HFC-1225ye and HFC-1225zc is one which causes the ratio of the relative volatilities of HFC-1225zc to HFC-1225ye to be greater than 1.1, with the HFC-1225zc being more volatile, thus permitting HFC-1225zc to be removed from the top of the distillation zone. Alternately, the appropriate extractive agent for a first mixture comprising HFC-1225ye and HFC-1225zc is one which causes the ratio of the relative volatilities of HFC-1225zc to HFC-1225ye to be less than 0.9, with the HFC-1225zc being less volatile, thus permitting HFC-1225ye to be recovered from the top of the distillation zone and HFC-1225zc to be removed from the bottom of the distillation zone together with the extractive agent. In order for an extractive agent to be effective in separating HFC-1225zc from HFC-1225ye by extractive distillation, the ratio of the relative volatilities of HFC-1225zc to HFC-1225ye in the presence of the extractive agent is greater than about 1.1 or less than about 0.9. Preferably, the ratio of the relative volatilities of HFC-1225zc to HFC-1225ye in the presence of the extractive agent is greater than about 1.3 or less than about 0.7, and still more preferably it is greater than about 1.5 or less than about 0.5.

In one embodiment of this invention, HFC-1225zc becomes more volatile than HFC-1225ye in the presence of the extractive agent, and is removed from the top of the distillation column. HFC-1225ye is recovered as a bottoms product together with extractive agent, and is further separated from the extractive agent in a conventional distillation column.

In another embodiment of this invention, HFC-1225ye becomes more volatile than HFC-1225zc in the presence of the extractive agent, and is recovered as pure product from the top of the distillation column. HFC-1225zc is removed from the bottom of the distillation column together with extractive agent.

In the extractive distillation process, the extractive agent is preferably recycled. For instance, for extractive agents causing HFC-1225zc more volatile than HFC-1225ye, extractive agent will be recovered from the bottom of the extraction column together with HFC-1225ye, and may be further purified in a conventional distillation column and recycled to the contacting step.

In one embodiment of this invention, the first mixture contains more than about 70 wt % of HFC-1225ye and that the HFC-1225zc content be less than about 30 wt %.

In another embodiment of this invention, the first mixture contains more than about 90 wt % of HFC-1225ye and that the HFC-1225zc content be less than about 10 wt %.

In another embodiment of this invention, the first mixture contains more than about 99 wt % of HFC-1225ye and that the HFC-1225zc content be less than about 1 wt %.

According to the present invention, HFC-1225ye containing less than 100 ppm of HFC-1225zc may be produced. Further, HFC-1225ye containing less than 10 ppm of HFC-1225zc, and even further HFC-1225ye containing less than 1 ppm of HFC-1225zc may be produced.

Also according to the present invention, HFC-1225ye containing less than 100 ppm of impurities may be produced. Further, HFC-1225ye containing less than 10 ppm of impurities may be produced, and even further, HFC-1225ye containing less than 1 ppm of impurities may be produced.

In one embodiment of the present process, alcohol extractive agent is introduced at an upper feed point of an extractive distillation column, whereas the first mixture comprising HFC-1225ye and HFC-1225zc is introduced at a relatively lower point in the column. The alcohol extractive agent passes downwardly through trays in the column and contacts the first mixture thereby forming a second mixture. While in the presence of the alcohol extractive agent, HFC-1225zc is relatively more volatile than HFC-1225ye, thereby causing overhead containing concentrated HFC-1225zc to exit the top of the column. Such overhead exiting the top of the column can be condensed by reflux condensers. At least a portion of this condensed overhead stream can be returned to the top of the column as reflux, and the remainder is either removed as waste or recovered as product. Alcohol extractive agent and HFC-1225ye comprise a third mixture that exits from the bottom of the column, which can then be passed to a stripper or distillation column for separation by using conventional distillation or other known methods. The alcohol extractive agent can be recycled to the extractive distillation column.

The ratio of the material exiting the top of the extractive distillation column, which is then condensed and in turn returned to the column, to the amount of remainder material that is removed or recovered is commonly referred to as the reflux ratio. The reflux ratio will define the physical characteristics of the extractive distillation column. For example, when THF or methanol is used as the extractive agent, an increase in the reflux ratio will in turn cause an increase of the HFC-1225ye recovery efficiency by reducing the quantity of HFC-1225ye in the overhead stream.

### EXAMPLES

The following Examples are provided to illustrate certain aspects of the present invention, and are not intended to limit the scope of the invention. The following Examples employ the NRTL equations identified above. In the following Examples, each stage is based upon a 100% operational or performance efficiency. In the following Examples, flow rates are given in pounds (weight)-per-hour (pph); temperatures are expressed in degrees Celsius (° C.); pressures are expressed in pound-per-square-inch-absolute (psia); stream concentrations are expressed in weight percentage (wt %) or parts-per-million-by-weight (ppm).

### COMPARATIVE EXAMPLE 1

In this Comparative Example, a crude feed stream comprising Z-HFC-1225ye and HFC-1225zc is fed to a distillation column operated under the four sets of conditions (cases) shown in Table 2, with the results of the distillations shown in the respective columns. The distillation columns in these Cases are operated to remove HFC-1225zc from the column as overhead distillate and a Z-HFC-1225ye product as column bottoms.

In Case 1 of this Comparative Example, a crude 100 pph Z-HFC-1225ye feed stream containing 5,000 ppm of HFC-1225zc is fed to a distillation column. The column has 104 stages and is 12 inches in diameter. As may be seen in this Case, when 1.5% of the crude feed to the column is taken overhead, the concentration of HFC-1225zc in the Z-HFC-1225ye bottoms product is only reduced to 385.9 ppm. Z-HFC-1225ye recovery efficiency is 99%.

Compared to Case 1, in Case 2 of this Comparative Example the column diameter has been increased to 16 inches, and distillate takeoff has been increased to 2.5% of the crude feed. The concentration of HFC-1225zc in the Z-HFC-1225ye bottoms product is reduced to 126.2 ppm.

But Z-HFC-1225ye recovery efficiency is also reduced to 98%.

Compared to Cases 1 and 2 above, in Case 3 of this Comparative Example the column diameter has been further increased to 23 inches, and distillate takeoff has been increased to 5.5% of the crude feed. The concentration of HFC-1225zc in the Z-HFC-1225ye bottoms product is now reduced to 34.8 ppm. But Z-HFC-1225ye recovery efficiency is also reduced to 95%.

Compared to Cases above, in Case 4 of this Comparative Example the column diameter has been further increased to 32 inches, and distillate takeoff has been increased to 10.5% of the crude feed. The concentration of HFC-1225zc in the Z-HFC-1225ye bottoms product is now reduced to 14.9 ppm. But Z-HFC-1225ye recovery efficiency is also reduced to 90%.

TABLE 2

| Case Number | 1 | 2 | 3 | 4 |
|---|---|---|---|---|
| # of total stages | 104 | 104 | 104 | 104 |
| Crude Feed Stage | 52 | 52 | 52 | 52 |
| Column Diameter (inch) | 12 | 16 | 23 | 32 |
| Distillate Temperature (° C.) | −7.7 | −7.4 | −7.1 | −7.0 |
| Bottoms Temperature (° C.) | −4.9 | −4.9 | −4.9 | −4.9 |
| Crude Feed Temperature | 10.0 | 10.0 | 10.0 | 10.0 |
| (° C.) | | | | |
| Top Pressure (psia) | 24.7 | 24.7 | 24.7 | 24.7 |
| Bottoms Pressure (psia) | 26.7 | 26.7 | 26.7 | 26.7 |
| Crude Feed Rate (pph) | 100 | 100 | 100 | 100 |
| Distillate Takeoff Rate (pph) | 1.5 | 2.5 | 5.5 | 10.5 |
| Bottoms Takeoff Rate (pph) | 98.5 | 97.5 | 94.5 | 89.5 |
| Reflux Rate (pph) | 2924 | 4975 | 10993 | 20997 |
| Feed to Column | | | | |
| Z-HFC-1225ye (wt %) | 99.5 | 99.5 | 99.5 | 99.5 |
| HFC-1225zc (wt %) | 0.5 | 0.5 | 0.5 | 0.5 |
| Distillate | | | | |
| HFC-1225zc (pph) | 0.46 | 0.49 | 0.50 | 0.50 |
| Z-HFC-1225ye Loss | 1.0 | 2.0 | 5.0 | 10.0 |
| Overhead (pph) | | | | |
| Z-HFC-1225ye in Feed | 1.0 | 2.0 | 5.0 | 10.0 |
| That Is Lost | | | | |
| Overhead (wt %) | | | | |
| Distillate from Column | | | | |
| Z-HFC-1225ye (wt %) | 68.4 | 80.4 | 91.0 | 95.2 |
| HFC-1225zc (wt %) | 31.6 | 19.6 | 9.0 | 4.8 |
| Bottoms from Column | | | | |
| Z-HFC-1225ye (wt %) | 100.0 | 100.0 | 100.0 | 100.0 |
| HFC-1225zc (ppm) | 385.9 | 126.2 | 34.8 | 14.9 |
| Z-HFC-1225ye Recovery | 99 | 98 | 95 | 90 |
| Efficiency (%) | | | | |

### Example 2

In this Example of the invention, THF is used as the extractive agent. A crude 100 pph Z-HFC-1225ye feed stream containing 5,000 ppm of HFC-1225zc is fed to an extractive distillation column. The extraction column has 62 stages and is 10 inches in diameter. As may be seen in Table 3 below, when 1.5% of the crude feed to the column is taken overhead, the concentration of HFC-1225zc in the Z-HFC-1225ye bottoms product is reduced to 10 ppm if THF extractant component is excluded. Z-HFC-1225ye recovery efficiency is 99%. The mixture of the bottoms product is then passed to a stripping column for separation by using conventional distillation. The stripping column has 32 stages and is 10 inches in diameter. As shown in Table 4 below, the distillate coming out from the top of the stripping column contains pure Z-HFC-1225ye product with only 10 ppm of HFC-1225zc impurity. The THF extractive agent coming from the bottom of the stripping column contains only trace amount of Z-HFC-1225ye and HFC-1225zc, and can be recycled to the extraction column.

TABLE 3
(Extraction Column)

| | |
|---|---|
| # of total stages | 62 |
| Crude Feed Stage | 27 |
| THF Feed Stage | 7 |
| Column Diameter (inch) | 10 |
| Distillate Temperature (° C.) | −7.7 |
| Bottoms Temperature (° C.) | 79.0 |
| Crude Feed Temperature (° C.) | 20.0 |
| THF Feed Temperature (° C.) | 10.4 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 2948.5 |
| Reflux Rate (pph) | 500 |
| THF Feed Rate (pph) | 2850 |
| THF in Distillate (pph) | 9E−10 |
| THF in Bottoms (pph) | 2850 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| THF (ppm) | 6E−4 |
| Bottoms from Column | |
| THF (wt %) | 96.66 |
| Z-HFC-1225ye (wt %) | 3.34 |
| HFC-1225zc (ppm) | 0.33 |
| Z-HFC-1225ye Recovery Efficiency (%) | 99.0 |

TABLE 4
(Stripping Column)

| | |
|---|---|
| # of total stages | 32 |
| Crude Feed Stage | 12 |
| Column Diameter (inch) | 10 |
| Distillate Temperature (° C.) | −6.9 |
| Bottoms Temperature (° C.) | 85.6 |
| Crude Feed Temperature (° C.) | 30.4 |
| Top Pressure (psia) | 24.7 |
| Bottoms Pressure (psia) | 26.7 |
| Crude Feed Rate (pph) | 2948.5 |
| Distillate Takeoff Rate (pph) | 98.5 |
| Bottoms Takeoff Rate (pph) | 2850 |
| Reflux Rate (pph) | 500 |
| Feed to Column | |
| THF (wt %) | 96.66 |
| Z-HFC-1225ye (wt %) | 3.34 |
| HFC-1225zc (ppm) | 0.33 |
| Distillate from Column | |
| Z-HFC-1225ye (wt %) | 99.999 |
| HFC-1225zc (ppm) | 9.88 |
| THF (ppm) | 3E−11 |
| Bottoms from Column | |
| THF (wt %) | 100.000 |
| Z-HFC-1225ye (ppm) | 2E−3 |
| HFC-1225zc (ppm) | 2E−13 |

### Example 3

In this Example of the invention, methanol is used as the extractive agent. A crude 100 pph Z-HFC-1225ye feed stream containing 5,000 ppm of HFC-1225zc is fed to an extractive distillation column. The extraction column has 62 stages and is 7 inches in diameter. As may be seen in Table 5 below, when 1.5% of the crude feed to the column is taken overhead, the concentration of HFC-1225zc in the Z-HFC-1225ye bottoms product is reduced to 10 ppm if methanol extractant component is excluded. Z-HFC-1225ye recovery efficiency is 99%. The mixture of the bottoms product is then passed to a stripping column for separation by using conventional distillation. The stripping column has 42 stages and is 7 inches in diameter. As shown in Table 6 below, the distillate coming out from the top of the stripping column contains pure Z-HFC-1225ye product with only 10 ppm of HFC-1225zc impurity. The methanol extractive agent coming from the bottom of the stripping column contains only trace amount of Z-HFC-1225ye and HFC-1225zc, and can be recycled to the extraction column.

TABLE 5
(Extraction Column)

| | |
|---|---|
| # of total stages | 62 |
| Crude Feed Stage | 25 |
| Methanol Feed Stage | 10 |
| Column Diameter (inch) | 7 |
| Distillate Temperature (° C.) | −7.7 |
| Bottoms Temperature (° C.) | 57.9 |
| Crude Feed Temperature (° C.) | 20.0 |
| Methanol Feed Temperature (° C.) | 10.3 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 1473.5 |
| Reflux Rate (pph) | 500 |
| Methanol Feed Rate (pph) | 1375 |
| Methanol in Distillate (pph) | 3E−5 |
| Methanol in Bottoms (pph) | 1375 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| Methanol (ppm) | 19.6 |
| Bottoms from Column | |
| Methanol (wt %) | 93.3 |
| Z-HFC-1225ye (wt %) | 6.7 |
| HFC-1225zc (ppm) | 0.67 |
| Z-HFC-1225ye Recovery Efficiency (%) | 99.0 |

TABLE 6
(Stripping Column)

| | |
|---|---|
| # of total stages | 42 |
| Crude Feed Stage | 30 |
| Column Diameter (inch) | 7 |
| Distillate Temperature (° C.) | −6.9 |
| Bottoms Temperature (° C.) | 80.5 |
| Crude Feed Temperature (° C.) | 30.3 |
| Top Pressure (psia) | 24.7 |
| Bottoms Pressure (psia) | 26.7 |
| Crude Feed Rate (pph) | 1473.5 |
| Distillate Takeoff Rate (pph) | 98.5 |
| Bottoms Takeoff Rate (pph) | 1375 |
| Reflux Rate (pph) | 500 |
| Feed to Column | |
| Methanol (wt %) | 93.3 |
| Z-HFC-1225ye (wt %) | 6.7 |
| HFC-1225zc (ppm) | 0.67 |
| Distillate from Column | |
| Z-HFC-1225ye (wt %) | 100.0 |
| HFC-1225zc (ppm) | 10.05 |
| Methanol (ppm) | 5E−3 |
| Bottoms from Column | |
| Methanol (wt %) | 100.0 |
| Z-HFC-1225ye (ppm) | 0.26 |
| HFC-1225zc (ppm) | 6E−8 |

### Example 4

In this Example of the invention, ethanol is used as the extractive agent. A crude 100 pph Z-HFC-1225ye feed stream containing 5,000 ppm of HFC-1225zc is fed to an extractive distillation column. The extraction column has 62 stages and is 7 inches in diameter. As may be seen in Table 7 below, when 1.5% of the crude feed to the column is taken overhead, the concentration of HFC-1225zc in the Z-HFC-1225ye bottoms product is reduced to 10 ppm if ethanol extractant component is excluded. Z-HFC-1225ye recovery efficiency is 99%. The mixture of the bottoms product is then passed to a stripping column for separation by using conventional distillation. The stripping column has 32 stages and is 8 inches in diameter. As shown in Table 8 below, the distillate coming out from the top of the stripping column contains pure Z-HFC-1225ye product with only 10 ppm of HFC-1225zc impurity. The ethanol extractive agent coming from the bottom of the stripping column contains only trace amount of Z-HFC-1225ye and HFC-1225zc, and can be recycled to the extraction column.

TABLE 7
(Extraction Column)

| | |
|---|---|
| # of total stages | 62 |
| Crude Feed Stage | 22 |
| Ethanol Feed Stage | 7 |
| Column Diameter (inch) | 7 |
| Distillate Temperature (° C.) | −7.7 |
| Bottoms Temperature (° C.) | 60.1 |
| Crude Feed Temperature (° C.) | 20.0 |
| Ethanol Feed Temperature (° C.) | 10.3 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 1673.5 |
| Reflux Rate (pph) | 500 |
| Ethanol Feed Rate (pph) | 1575 |
| Ethanol in Distillate (pph) | 2E−6 |
| Ethanol in Bottoms (pph) | 1575 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| Ethanol (ppm) | 1.46 |
| Bottoms from Column | |
| Ethanol (wt %) | 94.1 |
| Z-HFC-1225ye (wt %) | 5.9 |
| HFC-1225zc (ppm) | 0.59 |
| Z-HFC-1225ye Recovery Efficiency (%) | 99.0 |

TABLE 8
(Stripping Column)

| | |
|---|---|
| # of total stages | 32 |
| Crude Feed Stage | 12 |
| Column Diameter (inch) | 8 |
| Distillate Temperature (° C.) | −6.9 |
| Bottoms Temperature (° C.) | 94.2 |
| Crude Feed Temperature (° C.) | 30.3 |
| Top Pressure (psia) | 24.7 |
| Bottoms Pressure (psia) | 26.7 |
| Crude Feed Rate (pph) | 1673.5 |
| Distillate Takeoff Rate (pph) | 98.5 |
| Bottoms Takeoff Rate (pph) | 1575 |
| Reflux Rate (pph) | 500 |
| Feed to Column | |
| Ethanol (wt %) | 94.1 |
| Z-HFC-1225ye (wt %) | 5.9 |
| HFC-1225zc (ppm) | 0.59 |
| Distillate from Column | |
| Z-HFC-1225ye (wt %) | 100.0 |
| HFC-1225zc (ppm) | 10 |
| Ethanol (ppm) | 3E−3 |
| Bottoms from Column | |
| Ethanol (wt %) | 100 |
| Z-HFC-1225ye (ppm) | 7E−3 |
| HFC-1225zc (ppm) | 1.7E−10 |

### Example 5

In this Example of the invention, n-propanol is used as the extractive agent. A crude 100 pph Z-HFC-1225ye feed stream containing 5,000 ppm of HFC-1225zc is fed to an extractive distillation column. The extraction column has 62 stages and is 8 inches in diameter. As may be seen in Table 9 below, when 1.5% of the crude feed to the column is taken overhead, the concentration of HFC-1225zc in the Z-HFC-1225ye bottoms product is reduced to 10 ppm if n-propanol extractant component is excluded. Z-HFC-1225ye recovery efficiency is 99%. The mixture of the bottoms product is then passed to a stripping column for separation by using conventional distillation. The stripping column has 32 stages and is 9 inches in diameter. As shown in Table 10 below, the distillate coming out from the top of the stripping column contains pure Z-HFC-1225ye product with only 10 ppm of HFC-1225zc impurity. The n-propanol extractive agent coming from the bottom of the stripping column contains only trace amount of Z-HFC-1225ye and HFC-1225zc, and can be recycled to the extraction column.

TABLE 9
(Extraction Column)

| | |
|---|---|
| # of total stages | 62 |
| Crude Feed Stage | 22 |
| n-propanol Feed Stage | 7 |
| Column Diameter (inch) | 8 |
| Distillate Temperature (° C.) | −7.7 |
| Bottoms Temperature (° C.) | 64.8 |
| Crude Feed Temperature (° C.) | 20.0 |
| n-propanol Feed Temperature (° C.) | 10.3 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 2023.5 |
| Reflux Rate (pph) | 500 |
| n-propanol Feed Rate (pph) | 1925 |
| n-propanol in Distillate (pph) | 2.7E−9 |
| n-propanol in Bottoms (pph) | 1925 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| n-propanol (ppm) | 1.8E−3 |
| Bottoms from Column | |
| n-propanol (wt %) | 95.1 |
| Z-HFC-1225ye (wt %) | 4.9 |
| HFC-1225zc (ppm) | 0.49 |
| Z-HFC-1225ye Recovery Efficiency (%) | 99.0 |

TABLE 10
(Stripping Column)

| | |
|---|---|
| # of total stages | 32 |
| Crude Feed Stage | 12 |
| Column Diameter (inch) | 9 |
| Distillate Temperature (° C.) | −6.9 |
| Bottoms Temperature (° C.) | 114 |
| Crude Feed Temperature (° C.) | 30.3 |
| Top Pressure (psia) | 24.7 |
| Bottoms Pressure (psia) | 26.7 |
| Crude Feed Rate (pph) | 2023.5 |
| Distillate Takeoff Rate (pph) | 98.5 |
| Bottoms Takeoff Rate (pph) | 1925 |
| Reflux Rate (pph) | 500 |
| Feed to Column | |
| n-propanol (wt %) | 95.1 |
| Z-HFC-1225ye (wt %) | 4.9 |
| HFC-1225zc (ppm) | 0.49 |
| Distillate from Column | |
| Z-HFC-1225ye (wt %) | 100.0 |
| HFC-1225zc (ppm) | 10 |
| n-propanol (ppm) | 1.2E−8 |
| Bottoms from Column | |
| n-propanol (wt %) | 100 |
| Z-HFC-1225ye (ppm) | 3E−3 |
| HFC-1225zc (ppm) | 9E−11 |

### Example 6

In this Example of the invention, iso-propanol is used as the extractive agent. A crude 100 pph Z-HFC-1225ye feed stream containing 5,000 ppm of HFC-1225zc is fed to an extractive distillation column. The extraction column has 62 stages and is 8 inches in diameter. As may be seen in Table 11 below, when 1.5% of the crude feed to the column is taken overhead, the concentration of HFC-1225zc in the Z-HFC-1225ye bottoms product is reduced to 10 ppm if iso-propanol extractant component is excluded. Z-HFC-1225ye recovery efficiency is 99%. The mixture of the bottoms product is then passed to a stripping column for separation by using conventional distillation. The stripping column has 32 stages and is 9 inches in diameter. As shown in Table 12 below, the distillate coming out from the top of the stripping column contains pure Z-HFC-1225ye product with only 10 ppm of HFC-1225zc impurity. The iso-propanol extractive agent coming from the bottom of the stripping column contains only trace amount of Z-HFC-1225ye and HFC-1225zc, and can be recycled to the extraction column.

TABLE 11
(Extraction Column)

| | |
|---|---|
| # of total stages | 62 |
| Crude Feed Stage | 22 |
| Iso-propanol Feed Stage | 7 |
| Column Diameter (inch) | 8 |
| Distillate Temperature (° C.) | −7.7 |
| Bottoms Temperature (° C.) | 60.3 |
| Crude Feed Temperature (° C.) | 20.0 |
| Iso-propanol Feed Temperature (° C.) | 10.3 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 2038.5 |
| Reflux Rate (pph) | 500 |
| Iso-propanol Feed Rate (pph) | 1940 |
| Iso-propanol in Distillate (pph) | 4E−7 |
| Iso-propanol in Bottoms (pph) | 1940 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| Iso-propanol (ppm) | 0.27 |
| Bottoms from Column | |
| Iso-propanol (wt %) | 95.2 |
| Z-HFC-1225ye (wt %) | 4.8 |
| HFC-1225zc (ppm) | 0.49 |
| Z-HFC-1225ye Recovery Efficiency (%) | 99.0 |

TABLE 12
(Stripping Column)

| | |
|---|---|
| # of total stages | 32 |
| Crude Feed Stage | 12 |
| Column Diameter (inch) | 9 |
| Distillate Temperature (° C.) | −6.9 |
| Bottoms Temperature (° C.) | 98.3 |
| Crude Feed Temperature (° C.) | 30.3 |
| Top Pressure (psia) | 24.7 |
| Bottoms Pressure (psia) | 26.7 |
| Crude Feed Rate (pph) | 2038.5 |
| Distillate Takeoff Rate (pph) | 98.5 |
| Bottoms Takeoff Rate (pph) | 1940 |
| Reflux Rate (pph) | 500 |
| Feed to Column | |
| Iso-propanol (wt %) | 95.2 |
| Z-HFC-1225ye (wt %) | 4.8 |
| HFC-1225zc (ppm) | 0.49 |
| Distillate from Column | |
| Z-HFC-1225ye (wt %) | 100.0 |
| HFC-1225zc (ppm) | 10. |
| Iso-propanol (ppm) | 1E−4 |
| Bottoms from Column | |
| Iso-propanol (wt %) | 100.0 |
| Z-HFC-1225ye (ppm) | 3.5E−3 |
| HFC-1225zc (ppm) | 8.6E−11 |

### Example 7

In this Example of the invention, propylene oxide is used as the extractive agent. A crude 100 pph Z-HFC-1225ye feed stream containing 5,000 ppm of HFC-1225zc is fed to an extractive distillation column. The extraction column has 62 stages and is 12 inches in diameter. As may be seen in Table 13 below, when 1.5% of the crude feed to the column is taken overhead, the concentration of HFC-1225zc in the Z-HFC-1225ye bottoms product is reduced to 10 ppm if propylene oxide extractant component is excluded. Z-HFC-1225ye recovery efficiency is 99%. The mixture of the bottoms product is then passed to a stripping column for separation by using conventional distillation. The stripping column has 42 stages and is 9 inches in diameter. As shown in Table 14 below, the distillate coming out from the top of the stripping column contains pure Z-HFC-1225ye product with only 10 ppm of HFC-1225zc impurity. The propylene oxide extractive agent coming from the bottom of the stripping column contains only trace amount of Z-HFC-1225ye and HFC-1225zc, and can be recycled to the extraction column.

TABLE 13
(Extraction Column)

| | |
|---|---|
| # of total stages | 62 |
| Crude Feed Stage | 27 |
| Propylene oxide Feed Stage | 10 |
| Column Diameter (inch) | 12 |
| Distillate Temperature (° C.) | −7.7 |
| Bottoms Temperature (° C.) | 49.4 |
| Crude Feed Temperature (° C.) | 20.0 |
| Propylene oxide Feed Temperature (° C.) | 10.3 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 2598.5 |
| Reflux Rate (pph) | 2250 |
| Propylene oxide Feed Rate (pph) | 2500 |
| Propylene oxide in Distillate (pph) | 1.3E−9 |
| Propylene oxide in Bottoms (pph) | 2500 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| Propylene oxide (ppm) | 8.5E−4 |
| Bottoms from Column | |
| Propylene oxide (wt %) | 96.2 |
| Z-HFC-1225ye (wt %) | 3.8 |
| HFC-1225zc (ppm) | 0.39 |
| Z-HFC-1225ye Recovery Efficiency (%) | 99.0 |

TABLE 14
(Stripping Column)

| | |
|---|---|
| # of total stages | 42 |
| Crude Feed Stage | 12 |
| Column Diameter (inch) | 9 |
| Distillate Temperature (° C.) | −6.9 |
| Bottoms Temperature (° C.) | 52.3 |
| Crude Feed Temperature (° C.) | 30.3 |
| Top Pressure (psia) | 24.7 |
| Bottoms Pressure (psia) | 26.7 |
| Crude Feed Rate (pph) | 2598.5 |
| Distillate Takeoff Rate (pph) | 98.5 |
| Bottoms Takeoff Rate (pph) | 2500 |
| Reflux Rate (pph) | 1500 |
| Feed to Column | |
| Propylene oxide (wt %) | 96.2 |
| Z-HFC-1225ye (wt %) | 3.8 |
| HFC-1225zc (ppm) | 0.39 |
| Distillate from Column | |
| Z-HFC-1225ye (wt %) | 100.0 |
| HFC-1225zc (ppm) | 10 |
| Propylene oxide (ppm) | 9.7E−3 |
| Bottoms from Column | |
| Propylene oxide (wt %) | 100 |
| Z-HFC-1225ye (ppm) | 2.4E−2 |
| HFC-1225zc (ppm) | 7.9E−10 |

### Example 8

In this Example of the invention, 2-butanone (MEK) is used as the extractive agent. A crude 100 pph Z-HFC-1225ye feed stream containing 5,000 ppm of HFC-1225zc is fed to an extractive distillation column. The extraction column has 62 stages and is 12 inches in diameter. As may be seen in Table 15 below, when 1.5% of the crude feed to the column is taken overhead, the concentration of HFC-1225zc in the Z-HFC-1225ye bottoms product is reduced to 10 ppm if MEK extractant component is excluded. Z-HFC-1225ye recovery efficiency is 99%. The mixture of the bottoms product is then passed to a stripping column for separation by using conventional distillation. The stripping column has 32 stages and is 12 inches in diameter. As shown in Table 16 below, the distillate coming out from the top of the stripping column contains pure Z-HFC-1225ye product with only 10 ppm of HFC-1225zc impurity. The MEK extractive agent coming from the bottom of the stripping column contains only trace amount of Z-HFC-1225ye and HFC-1225zc, and can be recycled to the extraction column.

TABLE 15
(Extraction Column)

| | |
|---|---|
| # of total stages | 62 |
| Crude Feed Stage | 27 |
| MEK Feed Stage | 10 |
| Column Diameter (inch) | 12 |
| Distillate Temperature (° C.) | −7.7 |
| Bottoms Temperature (° C.) | 94.0 |
| Crude Feed Temperature (° C.) | 20.0 |
| MEK Feed Temperature (° C.) | 10.3 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 4198.5 |
| Reflux Rate (pph) | 500 |
| MEK Feed Rate (pph) | 4100 |
| MEK in Distillate (pph) | 3.4E−17 |
| MEK in Bottoms (pph) | 4100 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| MEK (ppm) | 2.3E−11 |
| Bottoms from Column | |
| MEK (wt %) | 97.7 |
| Z-HFC-1225ye (wt %) | 2.3 |
| HFC-1225zc (ppm) | 0.23 |
| Z-HFC-1225ye Recovery Efficiency (%) | 99.0 |

TABLE 16
(Stripping Column)

| | |
|---|---|
| # of total stages | 32 |
| Crude Feed Stage | 12 |
| Column Diameter (inch) | 12 |
| Distillate Temperature (° C.) | −6.9 |
| Bottoms Temperature (° C.) | 99.3 |
| Crude Feed Temperature (° C.) | 30.3 |
| Top Pressure (psia) | 24.7 |
| Bottoms Pressure (psia) | 26.7 |
| Crude Feed Rate (pph) | 4198.5 |
| Distillate Takeoff Rate (pph) | 98.5 |
| Bottoms Takeoff Rate (pph) | 4100 |
| Reflux Rate (pph) | 500 |
| Feed to Column | |
| MEK (wt %) | 97.7 |
| Z-HFC-1225ye (wt %) | 2.3 |
| HFC-1225zc (ppm) | 0.24 |
| Distillate from Column | |
| Z-HFC-1225ye (wt %) | 100.0 |
| HFC-1225zc (ppm) | 10 |
| MEK (ppm) | 2.9E−14 |
| Bottoms from Column | |
| MEK (wt %) | 100 |
| Z-HFC-1225ye (ppm) | 1.5E−3 |
| HFC-1225zc (ppm) | 8.7E−11 |

### Example 9

In this Example of the invention, diethyl ether (DEE) is used as the extractive agent. A crude 100 pph Z-HFC-1225ye feed stream containing 5,000 ppm of HFC-1225zc is fed to an extractive distillation column. The extraction column has 62 stages and is 12 inches in diameter. As may be seen in Table 17 below, when 1.5% of the crude feed to the column is taken overhead, the concentration of HFC-1225zc in the Z-HFC-1225ye bottoms product is reduced to 10 ppm if DEE extractant component is excluded. Z-HFC-1225ye recovery efficiency is 99%. The mixture of the bottoms product is then passed to a stripping column for separation by using conventional distillation. The stripping column has 42 stages and is 12 inches in diameter. As shown in Table 18 below, the distillate coming out from the top of the stripping column contains pure Z-HFC-1225ye product with only 10 ppm of HFC-1225zc impurity. The DEE extractive agent coming from the bottom of the stripping column contains only trace amount of Z-HFC-1225ye and HFC-1225zc, and can be recycled to the extraction column.

TABLE 17
(Extraction Column)

| | |
|---|---|
| # of total stages | 62 |
| Crude Feed Stage | 25 |
| DEE Feed Stage | 10 |
| Column Diameter (inch) | 12 |
| Distillate Temperature (° C.) | −7.7 |
| Bottoms Temperature (° C.) | 50.4 |
| Crude Feed Temperature (° C.) | 20.0 |
| DEE Feed Temperature (° C.) | 10.4 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 4298.5 |
| Reflux Rate (pph) | 500 |
| DEE Feed Rate (pph) | 4200 |
| DEE in Distillate (pph) | 2.2E−7 |
| DEE in Bottoms (pph) | 4200 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| DEE (ppm) | 0.15 |
| Bottoms from Column | |
| DEE (wt %) | 97.7 |
| Z-HFC-1225ye (wt %) | 2.3 |
| HFC-1225zc (ppm) | 0.23 |
| Z-HFC-1225ye Recovery Efficiency (%) | 99.0 |

TABLE 18
(Stripping Column)

| | |
|---|---|
| # of total stages | 42 |
| Crude Feed Stage | 15 |
| Column Diameter (inch) | 12 |
| Distillate Temperature (° C.) | −6.9 |
| Bottoms Temperature (° C.) | 52.4 |
| Crude Feed Temperature (° C.) | 30.4 |
| Top Pressure (psia) | 24.7 |
| Bottoms Pressure (psia) | 26.7 |
| Crude Feed Rate (pph) | 4298.5 |
| Distillate Takeoff Rate (pph) | 98.5 |
| Bottoms Takeoff Rate (pph) | 4200 |
| Reflux Rate (pph) | 1500 |
| Feed to Column | |
| DEE (wt %) | 97.7 |
| Z-HFC-1225ye (wt %) | 2.3 |
| HFC-1225zc (ppm) | 0.23 |
| Distillate from Column | |
| Z-HFC-1225ye (wt %) | 100.0 |
| HFC-1225zc (ppm) | 10 |
| DEE (ppm) | 3.1E−2 |
| Bottoms from Column | |
| DEE (wt %) | 100 |
| Z-HFC-1225ye (ppm) | 2.4 |
| HFC-1225zc (ppm) | 1.6E−8 |

### Example 10

In this Example of the invention, Toluene is used as the extractive agent. A crude 100 pph Z-HFC-1225ye feed stream containing 5,000 ppm of HFC-1225zc is fed to an extractive distillation column. The extraction column has 62 stages and is 11 inches in diameter. As may be seen in Table 19 below, when 1.5% of the crude feed to the column is taken overhead, the concentration of HFC-1225zc in the Z-HFC-1225ye bottoms product is reduced to 10 ppm if Toluene extractant component is excluded. Z-HFC-1225ye recovery efficiency is 99%. The mixture of the bottoms product is then passed to a stripping column for separation by using conventional distillation. The stripping column has 32 stages and is 14 inches in diameter. As shown in Table 20 below, the distillate coming out from the top of the stripping column contains pure Z-HFC-1225ye product with only 10 ppm of HFC-1225zc impurity. The Toluene extractive agent coming from the bottom of the stripping column contains only trace amount of Z-HFC-1225ye and HFC-1225zc, and can be recycled to the extraction column.

TABLE 19
(Extraction Column)

| | |
|---|---|
| # of total stages | 62 |
| Crude Feed Stage | 25 |
| Toluene Feed Stage | 7 |
| Column Diameter (inch) | 11 |
| Distillate Temperature (° C.) | −7.7 |
| Bottoms Temperature (° C.) | 109.0 |
| Crude Feed Temperature (° C.) | 20.0 |
| Toluene Feed Temperature (° C.) | 10.4 |
| Top Pressure (psia) | 24.7 |
| Bottoms Pressure (psia) | 26.7 |
| Crude Feed Rate (pph) | 100 |
| Distillate Takeoff Rate (pph) | 1.5 |
| Bottoms Takeoff Rate (pph) | 4298.5 |
| Reflux Rate (pph) | 500 |
| Toluene Feed Rate (pph) | 4200 |
| Toluene in Distillate (pph) | 2.1E−11 |
| Toluene in Bottoms (pph) | 4200 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.0 |
| Z-HFC-1225ye in Feed That Is Lost Overhead | 1.0 |
| (wt %) | |
| Distillate from Column | |
| Z-HFC-1225ye (wt %) | 66.7 |
| HFC-1225zc (wt %) | 33.3 |
| Toluene (ppm) | 1.4E−5 |
| Bottoms from Column | |
| Toluene (wt %) | 97.7 |
| Z-HFC-1225ye (wt %) | 2.3 |
| HFC-1225zc (ppm) | 0.23 |
| Z-HFC-1225ye Recovery Efficiency (%) | 99.0 |

TABLE 20
(Stripping Column)

| | |
|---|---|
| # of total stages | 32 |
| Crude Feed Stage | 12 |
| Column Diameter (inch) | 14 |
| Distillate Temperature (° C.) | −6.9 |
| Bottoms Temperature (° C.) | 133.0 |
| Crude Feed Temperature (° C.) | 30.4 |
| Top Pressure (psia) | 24.7 |
| Bottoms Pressure (psia) | 26.7 |
| Crude Feed Rate (pph) | 4298.5 |
| Distillate Takeoff Rate (pph) | 98.5 |
| Bottoms Takeoff Rate (pph) | 4200 |
| Reflux Rate (pph) | 500 |
| Feed to Column | |
| Toluene (wt %) | 97.7 |
| Z-HFC-1225ye (wt %) | 2.3 |
| HFC-1225zc (ppm) | 0.23 |
| Distillate from Column | |
| Z-HFC-1225ye (wt %) | 100.0 |
| HFC-1225zc (ppm) | 10 |
| Toluene (ppm) | 1.8E−13 |
| Bottoms from Column | |
| Toluene (wt %) | 100 |
| Z-HFC-1225ye (ppm) | 1.4E−3 |
| HFC-1225zc (ppm) | 4.3E−11 |

### Example 11

In this Example of the invention, fluorobenzene is used as the extractive agent. A crude 100 pph Z-HFC-1225ye feed stream containing 5,000 ppm of HFC-1225zc is fed to an extractive distillation column. The extraction column has 82 stages and is 12 inches in diameter. As may be seen in Table 21 below, when 2.0% of the crude feed to the column is taken overhead, the concentration of HFC-1225zc in the Z-HFC-1225ye bottoms product is reduced to 10 ppm if fluorobenzene extractant component is excluded. Z-HFC-1225ye recovery efficiency is 98.5%. The mixture of the bottoms product is then passed to a stripping column for separation by using conventional distillation. The stripping column has 32 stages and is 12 inches in diameter. As shown in Table 22 below, the distillate coming out from the top of the stripping column contains pure Z-HFC-1225ye product with only 10 ppm of HFC-1225zc impurity. The fluorobenzene extractive agent coming from the bottom of the stripping column contains only trace amount of Z-HFC-1225ye and HFC-1225zc, and can be recycled to the extraction column.

TABLE 21
(Extraction Column)

| | |
|---|---|
| # of total stages | 82 |
| Crude Feed Stage | 30 |
| Fluorobenzene Feed Stage | 7 |
| Column Diameter (inch) | 12 |
| Distillate Temperature (° C.) | −18.5 |
| Bottoms Temperature (° C.) | 83.0 |
| Crude Feed Temperature (° C.) | 20.0 |
| Fluorobenzene Feed Temperature (° C.) | 10.4 |
| Top Pressure (psia) | 15.7 |
| Bottoms Pressure (psia) | 17.7 |
| Crude Feed Rate (pph) | 100 |
| Distillate Takeoff Rate (pph) | 2.0 |
| Bottoms Takeoff Rate (pph) | 8598 |
| Reflux Rate (pph) | 1050 |
| Fluorobenzene Feed Rate (pph) | 8500 |
| Fluorobenzene in Distillate (pph) | 2.7E−9 |
| Fluorobenzene in Bottoms (pph) | 8500 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.5 |
| Z-HFC-1225ye in Feed That Is Lost Overhead | 1.5 |
| (wt %) | |
| Distillate from Column | |
| Z-HFC-1225ye (wt %) | 75.0 |
| HFC-1225zc (wt %) | 25.0 |
| Fluorobenzene (ppm) | 1.3E−3 |
| Bottoms from Column | |
| Fluorobenzene (wt %) | 98.86 |
| Z-HFC-1225ye (wt %) | 1.14 |
| HFC-1225zc (ppm) | 0.11 |
| Z-HFC-1225ye Recovery Efficiency (%) | 98.5 |

TABLE 22
(Stripping Column)

| | |
|---|---|
| # of total stages | 32 |
| Crude Feed Stage | 12 |
| Column Diameter (inch) | 12 |
| Distillate Temperature (° C.) | −6.9 |
| Bottoms Temperature (° C.) | 106.0 |
| Crude Feed Temperature (° C.) | 30.5 |
| Top Pressure (psia) | 24.7 |
| Bottoms Pressure (psia) | 26.7 |
| Crude Feed Rate (pph) | 8598.0 |
| Distillate Takeoff Rate (pph) | 98 |
| Bottoms Takeoff Rate (pph) | 8500 |
| Reflux Rate (pph) | 500 |
| Feed to Column | |
| Fluorobenzene (wt %) | 98.86 |
| Z-HFC-1225ye (wt %) | 1.14 |
| HFC-1225zc (ppm) | 0.113 |
| Distillate from Column | |
| Z-HFC-1225ye (wt %) | 100.0 |
| HFC-1225zc (ppm) | 10 |
| Fluorobenzene (ppm) | 5.1E−9 |
| Bottoms from Column | |
| Fluorobenzene (wt %) | 100 |
| Z-HFC-1225ye (ppm) | 7.1E−4 |
| HFC-1225zc (ppm) | 5.7E−10 |

### Example 12

In this Example of the invention, acetone is used as the extractive agent. A crude 100 pph Z-HFC-1225ye feed stream containing 5,000 ppm of HFC-1225zc is fed to an extractive distillation column. The extraction column has 82 stages and is 16 inches in diameter. As may be seen in Table 23 below, when 2.0% of the crude feed to the column is taken overhead, the concentration of HFC-1225zc in the Z-HFC-1225ye bottoms product is reduced to 49 ppm if acetone extractant component is excluded. Z-HFC-1225ye recovery efficiency is 98.5%. The mixture of the bottoms product is then passed to a stripping column for separation by using conventional distillation. The stripping column has 32 stages and is 17 inches in diameter. As shown in Table 24 below, the distillate coming out from the top of the stripping column contains pure Z-HFC-1225ye product with 49 ppm of HFC-1225zc impurity. The acetone extractive agent coming from the bottom of the stripping column contains only trace amount of Z-HFC-1225ye and HFC-1225zc, and can be recycled to the extraction column.

TABLE 23
(Extraction Column)

| | |
|---|---|
| # of total stages | 82 |
| Crude Feed Stage | 50 |
| Acetone Feed Stage | 7 |
| Column Diameter (inch) | 16 |
| Distillate Temperature (° C.) | −18.5 |
| Bottoms Temperature (° C.) | 60.1 |
| Crude Feed Temperature (° C.) | 20.0 |
| Acetone Feed Temperature (° C.) | 10.3 |
| Top Pressure (psia) | 15.7 |
| Bottoms Pressure (psia) | 17.7 |
| Crude Feed Rate (pph) | 100 |
| Distillate Takeoff Rate (pph) | 2.0 |
| Bottoms Takeoff Rate (pph) | 8598 |
| Reflux Rate (pph) | 500 |
| Acetone Feed Rate (pph) | 8500 |
| Acetone in Distillate (pph) | 3.1E−9 |
| Acetone in Bottoms (pph) | 8500 |
| Feed to Column | |
| Z-HFC-1225ye (wt %) | 99.5 |
| HFC-1225zc (wt %) | 0.5 |
| Distillate | |
| HFC-1225zc (pph) | 0.5 |
| Z-HFC-1225ye Loss Overhead (pph) | 1.5 |
| Z-HFC-1225ye in Feed That Is Lost Overhead | 1.5 |
| (wt %) | |
| Distillate from Column | |
| Z-HFC-1225ye (wt %) | 75.24 |
| HFC-1225zc (wt %) | 24.76 |
| Acetone (ppm) | 1.5E−3 |
| Bottoms from Column | |
| Acetone (wt %) | 98.86 |
| Z-HFC-1225ye (wt %) | 1.14 |
| HFC-1225zc (ppm) | 0.56 |
| Z-HFC-1225ye Recovery Efficiency (%) | 98.5 |

TABLE 24
(Stripping Column)

| | |
|---|---|
| # of total stages | 32 |
| Crude Feed Stage | 12 |
| Column Diameter (inch) | 17 |
| Distillate Temperature (° C.) | −6.9 |
| Bottoms Temperature (° C.) | 74.7 |
| Crude Feed Temperature (° C.) | 20.4 |
| Top Pressure (psia) | 24.7 |
| Bottoms Pressure (psia) | 26.7 |
| Crude Feed Rate (pph) | 8598.0 |
| Distillate Takeoff Rate (pph) | 98 |
| Bottoms Takeoff Rate (pph) | 8500 |
| Reflux Rate (pph) | 500 |
| Feed to Column | |
| Acetone (wt %) | 98.86 |
| Z-HFC-1225ye (wt %) | 1.14 |
| HFC-1225zc (ppm) | 0.56 |
| Distillate from Column | |
| Z-HFC-1225ye (wt %) | 100.0 |
| HFC-1225zc (ppm) | 49 |
| Acetone (ppm) | 6.8E−3 |
| Bottoms from Column | |
| Acetone (wt %) | 100 |
| Z-HFC-1225ye (ppm) | 9.2E−2 |
| HFC-1225zc (ppm) | 1.8E−7 |

What is claimed is:

1. A process for separating 1,2,3,3,3-pentafluoropropene from a first mixture comprising 1,2,3,3,3-pentafluoropropene and 1,1,3,3,3-pentafluoropropene, comprising the steps of:
a) contacting said first mixture with at least one extractive agent, to form a second mixture;
b) distilling said second mixture; and
c) recovering 1,2,3,3,3-pentafluoropropene substantially free of 1,1,3,3,3-pentafluoropropene.

2. The process of claim 1 wherein said at least one extractive agent is a compound having a normal boiling point between −10° C. and 120° C.

3. The process of claim 2 wherein said at least one extractive agent is a compound having a normal boiling point between 10° C. and 100° C.

4. The process of claim 3 wherein said at least one extractive agent is a compound having a normal boiling point between 30° C. and 70° C.

5. The process of claim 1, wherein said at least one extractive agent is selected from the group consisting of cyclic hydrocarbon ethers, non-cyclic hydrocarbon ethers, alcohols, toluene, fluorobenzene and ketones.

6. The process of claim 5, wherein the cyclic hydrocarbon ethers have from 2 to 6 carbon atoms.

7. The process of claim 5, wherein the noncyclic hydrocarbon ethers have the formula CₓH₂ₓ₊₁OC_{y}H_{2y+1} wherein x and y are 1 or greater and x+y is from 3 to 6.

8. The process of claim 5, wherein the alcohols have the formula C_{z}H_{2z+1}OH wherein z is from 1 to 4.

9. The process of claim 5, wherein the ketones have the formula CₘH₂ₘ₊₁C(O)CₙH₂ₙ₊₁ wherein m and n are 1 or greater and m+n is at most 5.

10. The process of claim 1 wherein said at least one extractive agent is selected from the group consisting of tetrahydrofuran, ethylene oxide, propylene oxide, oxetane, tetrahydropyran, diethyl ether, dipropyl ether, butyl methyl ether, methanol, ethanol, n-propanol, iso-propanol, toluene, fluorobenzene, acetone and 2-butanone.

11. The process of claim 10 wherein said at least one extractive agent is selected from the group consisting of methanol, ethanol, n-propanol, iso-propanol.

12. The process of claim 10 wherein said extractive agent is methanol.

13. The process of claim 10 wherein said extractive agent is selected from the group consisting of tetrahydrofuran and propylene oxide.

14. The process of claim 1 wherein the 1,2,3,3,3-pentafluoropropene recovered from the second mixture contains less than about 100 ppm of 1,1,3,3,3-pentafluoropropene.

15. The process of claim 1 wherein the 1,2,3,3,3-pentafluoropropene recovered from the second mixture contains less than about 10 ppm of 1,1,3,3,3-pentafluoropropene.

16. The process of claim 1 wherein the 1,2,3,3,3-pentafluoropropene recovered from the second mixture contains less than about 1 ppm of 1,1,3,3,3-pentafluoropropene.

17. The process of claim 1 wherein the volatility of said 1,1,3,3,3-pentafluoropropene or said 1,2,3,3,3-pentafluoropropene is increased, one relative to the other, in the presence of said at least one extractive agent.

18. The process of claim 1 wherein said 1,2,3,3,3-pentafluoropropene is Z-1,2,3,3,3-pentafluoropropene.

19. A composition of 1,2,3,3,3-pentafluoropropene containing less than about 100 ppm of 1,1,3,3,3-pentafluoropropene.

20. A composition of 1,2,3,3,3-pentafluoropropene containing less than about 100 ppm of impurities.
